(19) United States
(12) Patent Application Publication
(10) Pub. No.: US 2002/0052572 A1
(43) Pub. Date: May 2, 2002

(54) Resorbable anastomosis stents and plugs and their use in patients

(76) Inventors: Kenneth Franco, Omaha, NE (US); George Chu, Cupertino, CA (US); Frank A. DeLustro, Belmont, CA (US); George Y. Daniloff, Mountain View, CA (US)

Correspondence Address: REED & ASSOCIATES, 800 MENLO AVENUE, SUITE 210, MENLO PARK, CA, 94025, US

(21) Appl. No.: 09/966,800

(22) Filed: Sep. 25, 2001

Related U.S. Application Data
(60) Provisional application No. 60/235,036, filed on Sep. 25, 2000.
(60) Provisional application No. 60/259,997, filed on Jan. 5, 2001.

(51) Int. Cl. A61M 5/00

(52) U.S. Cl. 604/8

(57) ABSTRACT

The invention relates to anastomosis stents and plugs comprised of a non-polyglycolic acid material that is resorbable by the patient in about a few minutes up to about 90 days. Such stents and plugs may be employed in surgical techniques wherein tissue is joined at an interface without need for sutures, optionally through use of a tissue sealant. As a result, interfacial tensile strengths of at least about 1.3N/cm² may be achieved.

### CROSS-REFERENCE TO RELATED APPLICATIONS

This application claims priority under 35 U.S.C. §119(e)(1) to U.S. Provisional Patent Application Serial Nos. 60/235,036 and 60/259,997, filed on Sep. 25, 2000, and Jan. 5, 2001, respectively.

### TECHNICAL FIELD

The present invention generally relates to the anastomosis, or interconnection, between blood vessels or bodily tissues and to the covering of opening in tissues or blood vessels. More particularly, the invention pertains to stents and plugs, which are comprised of a material that is resorbable by a patient within a few minutes up to about 90 days.

### BACKGROUND

Over 15,000,000 people in the United States suffer from coronary artery disease, with approximately 500,000 new cases diagnosed each year, making coronary artery disease a significant national health problem. Symptomatic sufferers of coronary artery disease are often advised to undergo either percutaneous transluminal coronary angioplasty with stent implantation (PTCA/stent) or coronary artery bypass grafting (CABG). PTCA/stent, as a percutaneous procedure, is less invasive than open-heart surgery, although its effectiveness is limited due to the possible occurrence of arterial stent restenosis. The alternative procedure, CABG, performed with cardiopulmonary bypass or off-pump variants, requires an invasive incision that includes a median sternotomy for complete revascularization to bypass all three major coronary arteries.

Owing to the limitations of existing surgical interventions, there is a need to develop a closed-chest, totally endoscopic coronary artery bypass grafting procedure, which may be performed via a series of small incisions in the chest to gain access to the coronary arteries. As part of such an endoscopic procedure, the aorta or a coronary artery will be connected with a bypass conduit using a stent and sealing the anastomosis with a tissue sealant. Similarly, other surgical procedures would benefit from the use of a stent to join vessels and a tissue sealant to seal the resulting joint. In addition to coronary arteries, anastomosis of any artery, vein, the vas deferens, the fallopian tubes and any tissue with a lumen may benefit from such a stent and sealant.

Whereas traditional sutures and staples cinch together tissue to form a closure, a tissue adhesive allows for a tissue closure to retain the natural tissue orientation. Without adequate coverage around an opening in any tissue, the full advantages of tissue adhesives are not obtained. Thus, there exists a need for a plug capable of covering an opening in tissue to facilitate tissue adhesive closure. Similarly, because stents aid in holding vessel ends in a desired orientation during a surgical procedure and while vessel tissue is fused during healing, there is an ongoing need for improved stents.

Selection of materials is an important aspect of stent or plug construction. A number of suitable biocompatible materials have been developed that are based on collagenic materials, hydrophilic polymers, and conjugates thereof. See, e.g., U.S. Pat. Nos. 5,162,430, 5,324,775, 5,328,955, 5,470,911, 5,510,418, 5,550,188, and 5,565,519. Such materials are generally well suited for use in surgical and other techniques that require nonimmunogenic materials. One typical use for such materials is as an adhesive that serves to replace sutures or staples for surgery. These materials have also been employed to form flexible strings, see U.S. Pat. No. 5,308,889, to augment soft tissue in a mammal; see U.S. Pat. Nos. 5,306,500, 5,376,375, 5,413,791, 5,446,091 and 5,476,666, to repair bone defects; see U.S. Pat. No. 5,264,214 and to replace cartilage; see U.S. Pat. No. 5,304,595. In addition, such materials have been formed into tubes for use in vascular surgery. See U.S. Pat. No. 5,292,802 to Rhee et al.

Stents have been made from biological materials that are slowly resorbed by body tissue in the course of healing. Stent biological materials are usually polymeric and dissolve slowly over a period of weeks. A number of resorbable stent materials are described in U.S. Pat. Nos. 3,620,218, 3,683,926, 5,489,297, 5,653,744, and 5,762,625. Owing to the relatively slow resorption of the stents described in the prior art, the applications for resorbable stents have been limited. In addition, such stents are generally formed from materials containing polyglycolic acid, and the use of such materials may cause adverse tissue reactions. Thus, polyglycolic acid based stents may not be completely biocompatible for all patients.

U.S. Pat. No. 4,690,684 describes frozen blood plasma stents that are cylindrical masses lacking a fluid communicating bore. These stents are inserted into the interior of the ends of a tubular vessel to align the ends and to support the vessel during anastomosis. The stents are described only in terms of use in end-to-end vessel thermal bonding and present issues of sterility. In addition, as no fluid communicating bore is provided, these stents serve to occlude blood vessels for a period after the vessels have been joined and until the stents melt. The tendency of such stents to melt quickly renders them difficult to use. In addition, since these stents do not provide mechanical support to blood vessels once they have melted, these stents are incapable of providing support for more than an extremely short period.

Thus, there exists a need for a sterile, biocompatible, and resorbable stent capable of dissolution in the bloodstream, within about a few minutes up to about 90 days, that is useful in cardiac bypass procedures and other procedures requiring anastomosis. Similarly, there exists a need for resorbable plugs made from material similar to those used to for the resorbable stents as described above to cover opening in tissues or blood vessels.

### SUMMARY OF THE INVENTION

Accordingly, it is an object of the present invention to overcome the abovementioned disadvantages of the prior art by providing resorbable devices such as stents and plugs to support a bodily orifice or cavity during surgical techniques such as anastomosis.

It is another object of the invention to provide methods for using such stents and plugs in sutureless surgical techniques such as those that employ tissue sealants.

Additional objects, advantages and novel features of the invention will be set forth in part in the description that follows, and in part will become apparent to those skilled in the art upon examination of the following, or may be learned through routine experimentation upon practice of the invention.

In one embodiment, the invention relates to an anastomosis stent for insertion into an opening in a lumen of a vessel or tissue of a patient. The stent comprises: a first terminus; a second terminus; an opening at each terminus; and a primary lumen providing fluid communication between the openings at the first and second termini. At least one of the first and second termini is sized to be inserted into an opening in a vessel of a patient, and the stent is comprised of a non-polyglycolic acid material that is resorbable by the patient in about a few minutes up to about 90 days. Optionally, the stent further comprises a third terminus and a third opening at the third terminus, wherein the third opening is in fluid communication with the primary lumen through an intersecting lumen. While the dimensions and/or geometries of the stent may be selected according to intended use in various surgical techniques, at least one of the first and second termini typically is sized for anastomotic insertion into a blood vessel such as an artery or a vein of the patient.

The stent may be formed from one or more resorbable materials. In some embodiments, the material comprises frozen physiologic saline. In another embodiment, the material comprises a hydrophilic compound such as polyethylene glycol-containing compound or a collagenic material.

The inventive stent may be employed in a method of anastomosis comprising the steps of: inserting the first terminus of the stent though an aperture into the cavity of a physiologically functioning vessel of a patient, and the second terminus of the stent into a conduit, such that an interface is formed between the vessel and the conduit about the aperture; and attaching the vessel to the conduit at the interface. Alternatively, when the stent comprises a third terminus, the stent may be employed in a method of anastomosis comprising the steps of: inserting the first and second termini of the stent through in a physiologically functioning vessel of a patient, and the third terminus of the stent into a bypass conduit, such that an interface is formed between the vessel and the bypass conduit about the aperture; and attaching the vessel to the bypass conduit at the interface. Typically, the attachment is carried out without need for a suture such as by introducing a tissue sealant around or over the interface.

In another embodiment, the invention relates to a tissue plug for use in sealing an opening in a patient's tissue. The plug comprises a solid object having a platen surface, which is adapted to cover the opening, contact the perimeter about the opening, or both. The solid object is comprised of a non-polyglycolic acid material that is resorbable by the patient in a maximum of about 90 days. The plug may be comprises of any material suitable for forming the inventive stent.

The inventive plug may be employed in a method of sealing an opening in a patient's tissue. The method involves positioning the inventive plug in relationship to an opening in a patient's tissue, such that the plug covers the opening, contacts the perimeter about the opening, or both, thereby forming an interface between the plug and the tissue, and adhering the patient's tissue to the plug to form a closure. Typically, the patient's tissue is adhered to the plug through introducing a tissue sealant around or over the interface.

In still another embodiment, the invention relates to a sutureless method of anastomosis comprising the steps of: (a) providing a stent comprising a first terminus, a second terminus, a third terminus, an opening at each terminus that fluidly communicate with each other through the interior of the stent, wherein the stent is comprised of a non-polyglycolic acid material that is resorbable by a patient in up to about 90 days; (b) inserting the first and second termini of the stent though an aperture into a cavity of a physiologically functioning vessel of a patient, and the third terminus of the stent into a conduit, such that an interface is formed between the vessel and the by pass conduit about the aperture; and (c) applying a tissue sealant at the interface to attach the conduit to the vessel.

In a further embodiment, the invention relates to a sutureless method of sealing an opening in a patient's tissue comprising the steps of: (a) providing a plug comprised of a solid non-polyglycolic acid material that is resorbable by the patient in a maximum of about 90 days; (b) positioning the plug in relationship to an opening in a patient's tissue, such that the plug covers the opening, contacts the perimeter about the opening, or both, thereby forming an interface between the plug and the tissue; and (c) applying a resorbable sealant at the interface to form a closure.

In a still further embodiment, the invention relates to a sutureless method of anastomosis comprising the steps of: (a) providing a stent comprising a first terminus, a second terminus, a third terminus, an opening at each terminus that fluidly communicate with each other through the interior of the stent, wherein the stent is comprised of material that is resorbable by a patient in up to about 90 days; (b) inserting the first and second termini of the stent though an aperture into a cavity of a physiologically functioning vessel of a patient, and the third terminus of the stent into a conduit, such that an interface is formed between the vessel and the by pass conduit about the aperture; and (c) applying a tissue sealant at the interface to attach the conduit to the vessel such that the interface exhibits a tensile strength of at least about 1.3N/cm².

### BRIEF DESCRIPTION OF THE DRAWINGS

FIGS. 1A-1D, collectively referred to as FIG. 1, illustrate variations of the inventive stent. FIG. 1A illustrates an angled Y-shaped stent. FIG. 1B illustrates a partial Y-shaped stent similar to that illustrated in FIG. 1A, wherein the posterior portion of the primary cylindrical stent has been removed. FIG. 1C illustrates a partial T-shaped stent. FIG. 1D illustrates a cylindrical stent.

FIGS. 2A-2D, collectively referred to as FIG. 2, schematically illustrate the steps for conducting an anastomosis according to the present invention. FIG. 2A shows a vessel having an aperture formed by an incision through a side wall, the stent illustrated in FIG. 1C, and a bypass conduit. FIG. 2B shows the insertion of the flange portion of the stent into the incised vessel. FIG. 2C shows the insertion of an intersecting portion into the bypass conduit. FIG. 2D shows the completed anastomosis of the vessel and bypass conduit with tissue sealant.

FIGS. 3A-3D, collectively referred to as FIG. 3, illustrate various plugs of the invention.

FIGS. 4A-4E, collectively referred to as FIG. 4, are bar graphs relating to the swelling behavior of various stent materials.

### DETAILED DESCRIPTION OF THE INVENTION

Before the invention is described in detail, it is to be understood that unless otherwise indicated this invention is not limited to any particular materials, components, or manufacturing processes, as such may vary. It is also to be understood that the terminology used herein is for purposes of describing particular embodiments only, and is not intended to be limiting.

It must be noted that, as used in the specification and the appended claims, the singular forms “a,” “an”, and “the” include plural referents unless the context clearly dictates otherwise. Thus, for example, reference to “a stent” includes a single stent as well as two or more stents, “a lumen ” includes a single lumen as well as two or more lumens, and “a polymer” may encompass one or more polymers, and the like.

In this specification and in the claims that follow, reference will be made to a number of terms that shall be defined to have the following meanings unless the context clearly indicates otherwise:

The term “anastomosis” as used herein refers to the connection of separate or severed tubular hollow organs to form a continuous channel, as between two parts of the intestine or blood vessels.

The term “biocompatible” refers to the ability of the compositions of the present invention to be applied to tissues without eliciting significant inflammation, fibrosis, or tissue responses that are toxic, injurious or otherwise adverse.

The term “collagenic material” as used herein refers to all forms of collagen, including those that have been recombinantly produced, extracted, processed, or otherwise modified. Preferred collagens are non-immunogenic and, if extracted from animals, are treated to remove the immunogenic telopeptide regions (“atelopeptide collagen”), are soluble, and may be in the fibrillar or non-fibrillar form. Collagen used in connection with the preferred embodiments of the invention is in a pharmaceutically pure form such that it can be incorporated into a human body for the intended purpose.

The term “conjugated” is used herein to refer to attached through a chemical bond, typically a covalent bond.

The term “physiologic saline” as used herein refers to a substantially aqueous salt-containing solution conforming to normal, nonpathologic functioning of surrounding tissue and/or organs. For example, when physiologic saline is employed to form a stent for arterial anastomosis, the physiologic saline should be sterile and cannot contain pathogen of any type that will inhibit or interfere with arterial healing.

The term “polymer” refers to a molecule consisting of individual chemical moieties, which may be the same or different, but are preferably the same, that are joined together. As used herein, the term “polymer” refers to individual chemical moieties that are joined end-to-end to form a linear molecule, as well as individual chemical moieties joined together in the form of a branched structure.

The term “resorbable” is used herein in its ordinary sense and describes a material that can be both dissolved in and biologically assimilated by a patient.

The term “stent” is used herein in its ordinary sense and refers to a structure containing at least one lumen for insertion into a tubular structure, such as a blood vessel or an intestine, to provide support during or after the anastomosis.

The term “sealant”, as in “tissue sealant”, refers to compositions that become anchored in place by mechanical and/or chemical means to seal tissues together that have become separated as the result of various disease states or surgical procedures. For example, sealants can be used to fill voids in hard tissues, to join vascular and other soft tissues together, to provide a mechanical barrier to promote hemostasis, and to prevent tissue adhesions by keeping one tissue surface from coming in contact with and becoming adhered to another tissue surface. Unless the context clearly indicates otherwise, the term “sealant” is used interchangeably with the term “adhesive.”

The term “synthetic hydrophilic polymer” as used herein refers to a manmade polymer having an average molecular weight and composition that renders the polymer essentially water-soluble. Preferred polymers are highly pure or are purified to a highly pure state such that the polymer is, or is treated to become, pharmaceutically pure.

Thus, the invention generally relates to stents, plugs, and other solid articles that may be employed to provide mechanical support in surgical procedures such as anastomosis or to cover openings in tissues. The inventive articles are comprised of a material that is resorbable by a patient in about a few minutes to about 90 days. For example, the inventive article may be comprised of a sterile, biologically compatible substance capable of dissolution within the human body in less than a few hours or days. This is achieved through proper materials selection. In particular, the articles find use in endoscopic procedures performed in the abdomen or chest (such as coronary bypass grafting procedures that are performed through a series of small chest incisions to access coronary arteries).

In one embodiment, the invention provides an anastomotic stent for insertion into an opening in a vessel of a patient. The stent comprises a first terminus, a second terminus, and an opening at each terminus. A primary lumen extends from the first terminus to the second terminus thus providing fluid communication between the openings at the first and second termini. At least one of the first and second termini is sized for insertion into an opening in a vessel. The stent is comprised of a material that is resorbable by the patient in about a few minutes to about 90 days.

The stent may be employed in an anastomosis involving any of a number of vessels of a patient, including, but not limited to, blood vessels, including both arteries and veins; the intestines, including the small and/or large intestines; portions of the esophagus or trachea; urethra; fallopian tubes; vas deferens; eustachian tubes; lymph ducts; and/or virtually any channel within a living being, and specifically a channel of a human used to transport fluids or materials from one location to another within the body. Thus, the stent must be constructed according to the particular vessel or tissue in which the stent is to be inserted. For example, the inventive stent may be constructed for blood vessel anastomosis. In such a case, the stent must be sized and shaped according to the particular blood vessels to be joined in the anastomotic procedure. That is, the at least one of the first and second termini must be sized for anastomotic insertion into a blood vessel of the patient. In some instances, the lumen of the stent may be substantially straight. In other instances, the lumen may be curved, bent, or both. To facilitate stent insertion, at least one of the first and second termini may be tapered or otherwise shaped to exhibit a desired contour. Optionally both termini may be tapered. However, to constrain the stent within a vessel, the stent may further comprise a flange at one of the first and second termini.

For insertion into a small blood vessel, at least one of the first and second termini of the stent typically has an exterior diameter of about 1 mm to about 10 mm. Preferably, the diameter is about 1 mm to about 8 mm. Typically, internal bores of the stents have a diameter of less than about 0.5 to about 7 mm. When the stent is employed to join two blood vessels having approximately the same diameter, the first and second termini may have the same diameter. In the case wherein blood vessels having differing diameters are to be joined, it is preferred that the first and second termini have different diameters, the diameter of the termini selected according to the blood vessels to be joined.

In addition, the length of the stent should be selected according to the vessels to be joined. A stent having excessive length will be difficult to manipulate, whereas a stent having an inadequate length may not provide sufficient contact area for the stent to function as a structural support. Thus, when constructed for use in small blood vessel anastomoses, the inventive stent is usually about 1 cm to about 5 cm but preferably about 2 cm to about 3 cm in length.

Stents of the invention are generally produced with a smooth outer and inner surface. However, it is possible to produce the tubes so that the outer and/or inner surface(s) have any desired shape, such as an undulated surface. In some instances, it is possible to produce a tube that controllably increases or decreases in length by stretching or contracting the undulations of the tubular wall. In addition, the stent generally exhibits a circular cross-section along the length of the primary lumen, but may have any cross-sectional shape, including oval, square, triangular, hexagonal, etc.

The inventive stent may be employed to join two vessels. In such a case, the stent can be constructed as a tube having two termini, an opening at each terminus, and a lumen that provides communication between the openings. In some instances, however, the inventive stent may be employed in an anastomotic procedure to join additional vessels. Thus, although the stent walls are generally solid, openings may be provided for a variety of purposes. The inventive stent may further comprise an additional lumen branching from the lumen extending between the first and second termini. That is, an additional opening may be provided at a third terminus that fluidly communicates through an intersecting lumen with the lumen joining the openings at the first and second termini.

Depending on the intended purpose of the stent, the lumens may be joined in a number of ways. In some instances, the lumens may intersect at point closer to one of the first and second termini. In other instances, the branching lumen may be positioned at the midpoint between the first and second termini. While the lumens may intersect perpendicularly, it is more typical that the lumens intersect non-perpendicularly for blood vessel anastomosis. In some instances, the intersecting lumen may be initially provided as a separate component to be attached to the primary lumen. That is, the stents of the present invention may be formed by attaching a plurality of modular parts.

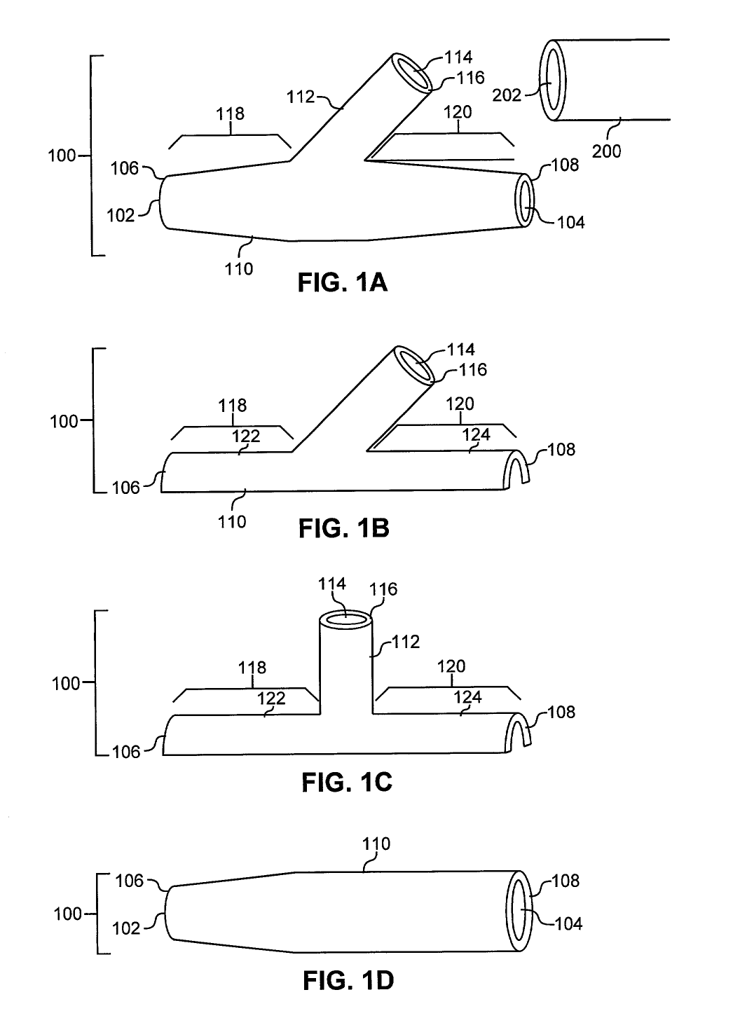

FIG. 1 illustrates various examples of the inventive stent. Each of the examples may be inserted within a blood vessel and a biological, or synthetic bypass conduit. As is the case with all figures referenced herein, in which like parts are referenced by like numerals, FIG. 1 is not necessarily to scale, and certain dimensions may be exaggerated for clarity of presentation. FIG. 1D illustrates a version of the inventive stent 100 according to the present invention having openings 102 and 104 located at the first terminus 106 and second terminus 108 of a substantially straight cylindrical portion 110. Located within the cylindrical portion 110 is a substantially straight primary lumen. This stent is particularly suited for use in forming an end-to-end joint between two vessels. While a two-ended stent may exhibit a uniform cross-sectional area along the length of the stent, the cylindrical stent 100 illustrated in FIG. 1D exhibits a tapered profile at the portion of the stent adjacent to terminus 106. As discussed above, such tapering facilitates insertion of terminus 106 into a vessel opening. In addition, this stent is particularly well suited for engaging two ducts of different luminal dimensions, terminus 106 for engaging a duct having a smaller luminal diameter than the duct to be engaged by terminus 108. Typically, the stent illustrated in FIG. 1D has an overall length between termini 106 and 108 of about 2 to about 3½ cm.

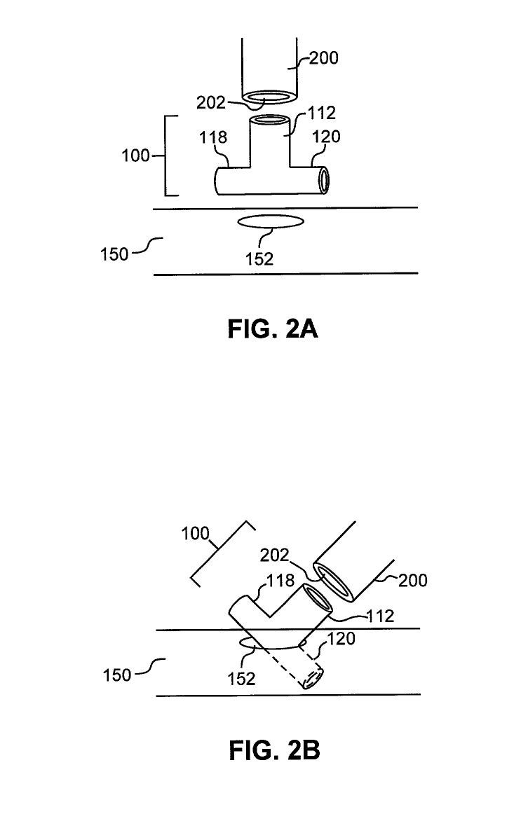

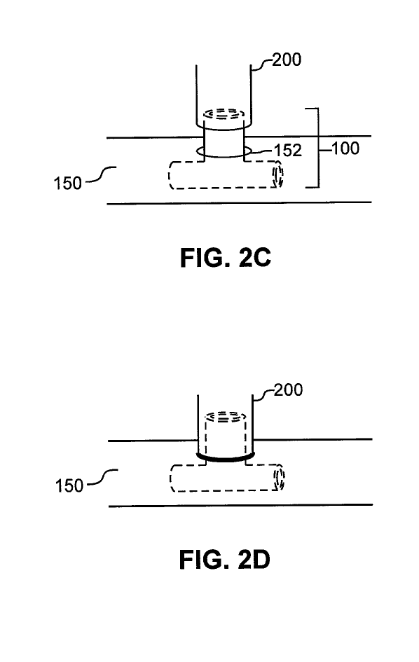

FIGS. 1A-1C illustrate stents having intersecting portions. FIG. 1A illustrates a Y-shaped stent 100. The Y-shaped stent is similar to the stent illustrated in FIG. 1D, except that it has three termini instead of two. That is, the stent 100 includes an intersecting portion 112 branching at a nonperpendicular angle from the primary cylindrical portion 110 between the first terminus 106 and the second terminus 108. The primary portion 110 may be adapted for insertion into the lumen 152 of a blood vessel 150 of FIG. 2. As illustrated, the intersecting portion 112 is also substantially cylindrical. An additional opening 114 is located at the terminus 116 of the intersecting portion 112 and is in fluid communication with the primary lumen through an intersecting lumen located within the intersecting section. As shown, the intersecting portion 112 joins the primary portion 110 at a point closer to terminus 108 than terminus 106. However, this is not a requirement; the intersecting portion may alternatively join the primary portion at a point closer to terminus 106 than to terminus 108, or at a point equidistant to termini 106 and 108, respectively. Thus, the intersecting portion 112 divides the primary cylindrical portion into two arms 118 and 120. It is appreciated that the dimensions of each arm 118 and 120, and the intersecting portion 112, are readily formed to engage a variety of vessel and/or conduit sizes. Typical dimensions for a stent, illustrated in FIG. 1A, for use in a coronary artery bypass procedure, are: for arm 118, a length of about 1 to about ½ cm, and for arm 120, a length of about ½ to about ¼ cm, each arm having an external diameter of about 1 to about 4 mm. In addition, intersecting portion 112 typically has a length of about 1½ to about 2½ cm and an outer diameter of about 1 to about 8 mm. Preferably, each of the arms 118 and 120 taper toward termini 106 and 108, respectively, to a smaller external diameter to facilitate insertion.

FIG. 1B illustrates another Y-shaped stent similar to that illustrated in FIG. 1A, except that the primary cylindrical portion has been substituted with a non-circumferential, partially cylindrical member that 110 having arms 118 and 120 terminating at termini 106 and 108, respectively. The partially cylindrical member 110 is shaped for insertion through an incision within a vessel such that the surfaces 122 and 124, associated with arms 118 and 120, respectively, generally conform to the lumenal dimensions of the blood vessel 150 of FIG. 2. Due to the geometry of the partially cylindrical member 110, insertion of this stent into a vessel causes less obstruction as compared to insertion of the stent depicted in FIG. 1A.

FIG. 1C illustrates a stent similar to that illustrated in FIG. 1B, except that the intersecting portion 112 extends perpendicularly from the partially cylindrical member 110. Thus, a T-shaped stent is formed. Like the stent illustrated in FIG. 2B, this stent is also well suited for an aortic anastomotic procedure. As shown, the stent 100 has two arms 118 and 120 on either side of the intersecting portion 112. Again, it is preferred that the terminus 116 of the intersecting portion 112, and the arms 118 and 120, are tapered to facilitate insertion within a bypass conduit or vessel. Typical dimensions for a stent, illustrated in FIG. 1C, for use in a coronary artery bypass procedure, are: for arm 118, a length of about 1 to about 2 centimeters, and for arm 120, a length of about ½ to about 1 cm, each arm having an external diameter of about 8 to about 11 mm. In addition, intersecting portion 112 typically has a length of about 1½ to about 2½ cm and an outer diameter of about 1 to about 8 mm. Preferably, the intersecting portion 112 has a length greater than either of arms 118 and 120.

The stent described above may be employed to carry out an inventive method for carrying out an anastomosis. When the stent only has two termini, the method involves inserting the first terminus of the inventive stent though an aperture into the opening of a physiologically functioning vessel of a patient. The second terminus of the stent is inserted into a conduit such that an interface is formed between the vessel and the conduit about the aperture. When the stent comprises three termini, the method involves inserting the first and second termini of the inventive stent though an aperture into the opening of a physiologically functioning vessel of a patient. The third terminus of the stent is inserted into a bypass conduit such that an interface is formed between the vessel and the bypass conduit about the aperture. In either case, the vessel is attached to the conduit at the interface, either as the stent is being inserted into the conduit and the vessel, or after insertion. While attachment may be carried out using a variety of means, e.g., using sutures, staples, etc., it is preferred that the vessel and the conduit be attached without need for a suture. Typically, this involves introducing a tissue sealant into the interface between the vessel and the conduit. For example, the sealant may be spread around or sprayed over the interface. In addition, the sealant may be provided on any surface of the inventive stent that may come into contact with another surface, e.g., tissue surface, lumen surface. Thus, a sealant may be provided on the exterior surface of the inventive stent. The sealant can be provided as a contiguous or noncontiguous coating in solid, gel or liquid form. In some instances, the sealant may be provided as a dry powder that becomes activated upon contact with a liquid such as that present during typical anastomotic procedures. In addition or in the alternative, the stent itself may be formed from a material compounded with one or more sealants. A number of sealants are known in the art (see infra); preferred sealants include collagenic materials, polyethylene glycols, mixtures thereof, and copolymers thereof. Optionally, the sealant may be crosslinked after application at the interface.

FIG. 2 illustrates the steps for performing an anastomosis according to the present invention. As illustrated in FIG. 2A, a blood vessel 150 is provided having a sidewall aperture 152. The blood vessel is adapted to be connected to conduit 200 though blunt end 202 by way of the stent 100 as shown in FIG. 1C. In FIG. 2B, an arm 120 is inserted through the aperture 152 in the vessel 150 with an angular motion relative to the walls of the vessel 150. The stent 100 is then pulled against the vessel sidewalls defining the aperture 152 until arm 118 also enters the vessel 150 through aperture 152. Depending on the material employed to form the inventive stent, the stent may be elastically or plastically deformed during insertion. As illustrated in FIG. 2C, the blunt cut end 202 of conduit 200 is engaged with the intersecting portion 112 of the stent 100. That is, conduit 200 is slipped over the intersecting portion 112 towards the vessel 150. Excessive blood and moisture are removed from the region around the aperture 152 and a tissue adhesive is applied about the aperture 152 and/or the end 202 of conduit 200 as the conduit 200 is brought into physical contact with the vessel 150. The tissue sealant includes collagen-containing tissue adhesives that exhibit a bond strength comparable to that formed from polymerizing alkyl cyanoacrylate monomers as well as other compositions discussed infra. After the tissue adhesive is contacted with the vessel 150 and conduit 200 for few minutes, a seal is formed at the interface, as shown in FIG. 2D. With the fairly rapid dissolution of a stent according to the present invention, the integrity of the resulting tissue adhesive joint is readily monitored during the course of the surgical procedure thereby allowing for correction of seepage.

Thus, the invention also provides a sutureless method of anastomosis. In some instances, a stent is provided comprising a first terminus, a second terminus, and an opening at each terminus that fluidly communicate through a lumen therebetween. The first terminus of the stent is inserted through an aperture into an opening cavity of a physiologically functioning vessel of a patient, and the second terminus of the stent is inserted into a conduit such that an interface is formed between the vessel and the conduit about the aperture. When the stent further comprises a third terminus having an opening that fluidly communicates with the lumen, the first and second termini of the stent is inserted through an aperture into an opening cavity of a physiologically functioning vessel of a patient, and the third terminus of the stent is inserted into a bypass conduit such that an interface is formed between the vessel and the bypass conduit about the aperture. In either case, the stent is comprised of a non-polyglycolic acid material that is resorbable by the patient in a few minutes up to about to about 90 days. The method is completed when a tissue sealant is applied at the interface to attach the conduit to the vessel.

In another embodiment, the invention provides a tissue plug for use in covering an opening in a patient's tissue. The plug may be employed, for example, to cover an opening in a vessel or tissue or to facilitate the use of a tissue sealant to close the opening. As used herein “opening” as in a “tissue opening” refers to any cut, tear, laceration or fissure in any living tissue. The inventive plug comprises a solid object having a platen surface and is adapted to cover the opening, contact the perimeter about the opening, or both. As is the case with the inventive stent, the solid object is comprised of a non-polyglycolic acid material that is resorbable by the patient in no more than about 90 days. The plug is particularly useful in providing a dry field (preventing further leakage of blood, etc.) until a tissue sealant can be applied to form a closure.

The plug may be formed into any shape suitable for its intended use. For example, the platen surface may be supported by a pedestal structure having a pedestal lateral dimension. In some instances, the platen surface may have a lateral dimension equal to the pedestal structure lateral dimension. In other instances, the platen surface may be formed to exhibit a lateral dimension greater than the pedestal structure lateral dimension. The platen surface is nonplanar, e.g., to facilitate the conformation of the platen surface to the lumen surface to effect the sealing of openings in tissues such as blood vessels, intestines, the stomach, and other fluid ducts including hepatic, bile, tear, cranial, seminal, and the like. In a preferred embodiment, the inventive plug may be employed during surgery involving a blood vessel such as an artery or vein. Depending on the surgery needed, the plug may be employed in surgery involving a coronary artery or the aorta of a patient.

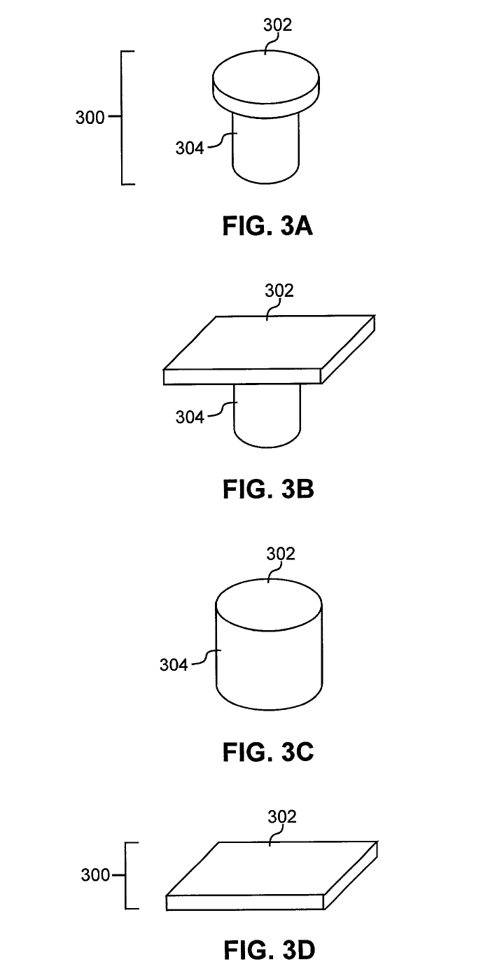

FIG. 3 illustrates various inventive plugs. FIG. 3A, for example, illustrates a plug 300 having a substantially circular platen surface 302 and a cylindrical supporting structure 304. FIG. 3B illustrated a plug similar to that illustrated in FIG. 3A, except that the platen surface 302 is rectangular. FIG. 3C illustrates a plug similar to that illustrated in FIGS. 3A and 3B, except that the platen surface 302 is identically sized to the cross-section of the supporting structure. While the plugs illustrated in FIGS. 3A-3C are depicted having a supporting portion 304 as being generally columnar in shape, it is appreciated that a variety of support structure shapes are operative. It is also appreciated that the relative size and shape of the platen relative to the base portion of a plug is variable to accommodate closing of openings within a variety of tissues. For example, FIG. 3D illustrates a plug 300 formed from a planar or a substratum-conforming platen 302 that can be laid over an opening in the tissue. This tissue flap closure plug 300 thus functions independent of a pedestal portion.

The inventive plug may be employed to seal an opening in a patient's tissue. Thus, an inventive method is provided wherein the inventive plug is positioned in relationship to an opening in a patient's tissue such that the plug covers the opening, contacts the perimeter about the opening, or both. As a result, an interface is formed between the plug and the tissue. The patient's tissue is adhered to the plug to form a closure.

Similar to the inventive method for carrying out an anastomosis, the closure is formed by introducing a tissue sealant onto the interface. While attachment may be carried out using a variety of means, e.g., using sutures, staples, etc., it is preferred that the opening in the tissue will be closes without need for a suture. The sealant may be injected around or applied as a spray over the interface as is the case with the inventive stent. Likewise, the sealant may be provided on any surface of the inventive plug that may come into contact with another surface. The same tissue sealants that may be used for anastomosis may be employed when using a plug to seal a tissue opening. When a plug as illustrated in FIG. 3D is employed, additional tissue may be placed in contact with the plug such that the plug is interposed between the additional tissue and the tissue associated with the opening. Optionally, the additional tissue may be adhered to the tissue associated with the opening.

Thus, another embodiment of the invention relates to a sutureless method of sealing an opening in a patient's tissue. A plug is provided that comprises a solid non-polyglycolic acid material that is resorbable by the patient in no more than about 90 days. The plug is positioned in relationship to an opening in a patient's tissue such that the plug covers the opening, contacts the perimeter about the opening, or both, thereby forming an interface between the plug and the tissue. To form the closure, a tissue sealant is applied at the interface.

In general, the inventive stents and plugs may be formed from any of a number of nonpolyglycolic acid materials to allow for resorption in about a few minutes to about 90 days. All suitable materials are non-toxic, noninflammatory and nonimmunogenic when used to form the stents and plugs of the invention. Typically, the material is resorbable by the patient in about one to about ten days. In instances where the stent is needed to promote healing for a relatively extended period of time, the material may be selected such that the stent is resorbed by the patient in about seven to about ten days. In other instances, the material may be selected such that the stent is resorbed by the patient in about one to about seven days, optimally in about one to about two days.

In order to construct stents that are resorbed in a short period of time, materials comprising frozen physiologic saline may be employed. More typically, materials comprising a hydrophilic compound are employed. Often, polymeric materials are employed because the resorption rate may be established by controlling the molecular weight and/or the degree of crosslinking associated with the polymeric material. In general, hydrophilic polymers can be rendered water-soluble by incorporating a sufficient number of oxygen (or less frequently nitrogen) atoms available for forming hydrogen bonds in aqueous solutions. Suitable hydrophilic polymers used herein include polyethylene glycol, polyoxyethylene, polymethylene glycol, polytrimethylene glycols, polyvinylpyrrolidones, and derivatives thereof. In some limited instances, polylactic acids may be employed as well. The polymers can be linear or multiply branched and will not be substantially crosslinked. Other suitable polymers include polyoxyethylene-polyoxypropylene block polymers and copolymers. Polyoxyethylene-polyoxypropylene block polymers having an ethylene diamine nucleus (and thus having four ends) are also available and may be used in the practice of the invention.

One preferred material for use in the present invention comprises a polyethylene glycol (PEG) containing compound, due to its known biocompatibility. Various forms of PEG are extensively used in the modification of biologically active molecules because PEG can be formulated to have a wide range of solubilities and because it is low in toxicity, antigenicity, immunogenicity, and does not typically interfere with the enzymatic activities and/or conformations of peptides. Further, PEG monomers are generally non-biodegradable and is easily excreted from most living organisms, including humans.

Suitable PEGs include mono-, di-, and multifunctional PEG. Monofunctional PEG has only one reactive hydroxy group, while difunctional PEG has reactive groups at each end. Monofunctional PEG preferably has an average molecular weight between about 100 and about 15,000 daltons, more preferably between about 200 and about 8,000, and most preferably about 4,000. Difunctional and multifunctional PEG preferably have a molecular weight of about 400 to about 100,000, more preferably about 3,000 to about 20,000.

Those of ordinary skill in the art will appreciate that synthetic polymers such as PEG cannot be prepared practically to have exact molecular weights, and that the term “molecular weight” as used herein refers to an average molecular weight of a number of molecules in any given sample, as commonly used in the art. Thus, a sample of PEG 2,000 might contain a statistical mixture of polymer molecules ranging in weight from, for example, 1,500 to 2,500 daltons, with one molecule differing slightly from the next over a range. Specification of a range of molecular weight indicates that the average molecular weight may be any value between the limits specified, and may include molecules outside those limits. Thus, a molecular weight range of about 800 to about 20,000 indicates an average molecular weight of at least about 800, ranging up to about 20 kDa.

PEG can be rendered monofunctional by forming an alkylene ether at one end. The alkylene ether may be any suitable alkoxy radical having 1-6 carbon atoms, for example, methoxy, ethoxy, propoxy, 2-propoxy, butoxy, hexyloxy, and the like. Methoxy is presently preferred. Difunctional PEG is provided by allowing a reactive hydroxy group to exist at each end of the linear molecule. The reactive groups are preferably at the ends of the polymer, but may be provided along the length thereof. Polyfunctional molecules are capable of crosslinking the compositions of the invention, and may be used to attach additional moieties.

In some instances, naturally occurring compounds may be employed as stent or plug material. Suitable naturally occurring compounds include, but are not limited to: polysaccharides such as hyaluronic acid, cyclodextrin, hydroxymethylcellulose, cellulose ether, and starch; glycans such glycosaminoglycan and proteoglycan; and various proteins. Proteins such as collagen and other collagenic materials are particularly suited for use in the present invention.

It is known in the art that collagen is the major protein component of bone, cartilage, skin, and connective tissue in animals. Collagen, in its native form, is typically a rigid, rod-shaped molecule approximately 300 nm long and 1.5 nm in diameter. It is composed of three collagen polypeptides, which together form a tight triple helix. The collagen polypeptides are each characterized by a long midsection having the repeating sequence -Gly-X-Y-, where X and Y are often proline or hydroxyproline, bounded at each end by the “telopeptide” regions, which constitute less than about 5% of the molecule. The telopeptide regions of the collagen chains are typically responsible for the crosslinking between chains, and for the immunogenicity of the protein. Collagen occurs in several types, having distinct physical properties. The most abundant types are Types I, II and III. Further, collagen is typically isolated from natural sources, such as bovine hide, cartilage, or bones. Bones are usually dried, defatted, crushed, and demineralized to extract collagen, while hide and cartilage are usually minced and digested with proteolytic enzymes (other than collagenase). As collagen is resistant to most proteolytic enzymes, this procedure conveniently serves to remove most of the contaminating protein found with collagen.

Suitable collagenic materials include all types of pharmaceutically useful collagen, preferably types I, II, and III. Collagens may be soluble (for example, commercially available Vitrogen® 100 collagen-in-solution), and may or may not have the telopeptide regions. Preferably, the collagen will be reconstituted fibrillar atelopeptide collagen, for example Zyderm® collagen implant (ZCI) or atelopeptide collagen in solution (CIS). Optionally, colony stimulating factors (CSFs) may be included as well. Various forms of collagen are available commercially, or may be prepared by the processes described in, for example, U.S. Pat. Nos. 3,949,073, 4,488,911, 4,424,208, 4,582,640, 4,642,117, 4,557,764, and 4,689,399. In addition, other forms of collagen are also useful in the practice of the invention, and are not excluded from consideration here. For example, non-fibrillar collagens such as methylated or succinylated collagens may be employed in the present invention. In some instances, collagen crosslinked using heat, radiation, or chemical agents such as glutaraldehyde may be employed. Similarly, gelatin, i.e., collagen denatured typically through boiling, may be suitable.

The inventive stents and plugs may be formed from any of the aforementioned materials singularly or in combination. In some instances, conjugates of the aforementioned materials may be employed. For example, collagenic material may be chemically bound to a synthetic hydrophilic polymer. The chemical binding can be carried out in a variety of ways. In accordance with the preferred method, the synthetic hydrophilic polymer is activated and then reacted with the collagen. Alternatively, the hydroxyl or amino groups present on the collagen can be activated, and the activated groups reacted with the polymer to form the conjugate. In accordance with a less preferred method, a linking group with activated hydroxyl or amino groups thereon can be combined with the polymer and collagen in a manner so that it will concurrently react with both the polymer and collagen, forming the conjugate. Since the inventive stents and plugs are to be used in the human body, it is important that all of the components of the conjugate, e.g., polymer, collagen, and linking group, singly and in combination, are unlikely to be rejected by the body. Accordingly, toxic and/or immunoreactive components are not preferred as starting materials.

For example, the first step in forming the collagen-polymer conjugates often involves the functionalization of the polymer molecule. Various functionalized PEGs have been used effectively in fields such as protein modification (see Abuchowski et al., Enzymes as Drugs, John Wiley & Sons: New York, N.Y. (1981) pp. 367-383; and Dreborg et al., Crit. Rev. Therap. Drug Carrier Syst. (1990) 6:315, both of which are incorporated herein by reference), peptide chemistry (see Mutter et al., The Peptides, Academic: New York, N.Y. 2:285-332; and Zalipsky et al., Int. J. Peptide Protein Res. (1987) 30:740, both of which are incorporated herein by reference), and the synthesis of polymeric drugs (see Zalipsky et al., Eur. Polym. J. (1983) 19:1177; and Ouchi et al., J. Macromol. Sci. -Chem. (1987) A24:1011. Various types of conjugates formed by the binding of PEG with specific, pharmaceutically active proteins have been disclosed and found to have useful medical applications, in part due to the stability of such conjugates with respect to proteolytic digestion, reduced immunogenicity, and longer half-lives within living organisms.

One form of PEG that has been found to be particularly useful is monomethoxypolyethylene glycol (mPEG), which can be activated by the addition of a compound such as cyanuric chloride, then coupled to a protein (see Abuchowski et al., J. Biol. Chem. (1977) 252:3578, which is incorporated herein by reference). Although such methods of activating PEG can be used in connection with the present invention, they are not particularly desirable in that the cyanuric chloride is relatively toxic and must be completely removed from any resulting product in order to provide a pharmaceutically acceptable composition.

Activated forms of PEG can be made from reactants that can be purchased commercially. One form of activated PEG, which has been found to be particularly useful in connection with the present invention, is mPEG-succinate-N-hydroxysuccinimide ester (SS-PEG) (see Abuchowski et al., Cancer Biochem. Biphys. (1984) 7:175, which is incorporated herein by reference). Activated forms of PEG such as SS-PEG react with the proteins under relatively mild conditions and produce conjugates without destroying the specific biological activity and specificity of the protein attached to the PEG. However, when such activated PEGs are reacted with proteins, they react and form linkages by means of ester bonds. Although ester linkages can be used in connection with the present invention, they are not particularly preferred in that they undergo hydrolysis when subjected to physiological conditions over extended periods of time (see Dreborg et al., Crit. Rev. Therap. Drug Carrier Syst. (1990) 6:315; and Ulbrich et al., J. Makromol. Chem. (1986) 187:1131, both of which are incorporated herein by reference).

It is possible to link PEG to proteins via urethane linkages, thereby providing a more stable attachment that is more resistant to hydrolytic digestion than the ester linkages (see Zalipsky et al., Polymeric Drug and Drug Delivery Systems, Chapter 10, “Succinimidyl Carbonates of Polyethylene Glycol” (1991) incorporated herein by reference to disclose the chemistry involved in linking various forms of PEG to specific biologically active proteins). The stability of urethane linkages has been demonstrated under physiological conditions (see Veronese et al., Appl. Biochem. Biotechnol. (1985) 11:141; and Larwood et al., J. Labelled Compounds Radiopharm. (1984) 21:603, both of which are incorporated herein by reference). Another means of attaching the PEG to a protein can be by means of a carbamate linkage (see Beauchamp et al., Anal. Biochem. (1983) 131:25; and Berger et al., Blood (1988) 71:1641, both of which are incorporated herein by reference). The carbamate linkage is created by the use of carbonyldiimidazole-activated PEG. Although such linkages have advantages, the reactions are relatively slow and may take 2 to 3 days to complete.

The conjugates formed using the functionalized forms of PEG vary depending on the functionalized form of PEG that is used in the reaction. Furthermore, the final product can be modified with respect to its characteristics by changing the molecular weight of the PEG. In general, the stability of the conjugate is improved by eliminating any ester linkages between the PEG and the collagen, and including ether and/or urethane linkages. However, to promote resorption, weaker ester linkages may be included so that the linkages are gradually broken by hydrolysis under physiological conditions. That is, by varying the chemical structure of the linkage, the rate of resorption can be varied.

Polyfunctional polymers may also be used to crosslink collagen molecules to other proteins (e.g., glycosaminoglycans, chondroitin sulfates, fibronectin, and the like), particularly growth factors, for compositions particularly suited for use in wound healing, osteogenesis, and immune modulation. Such tethering of cytokines to collagen molecules provides an effective slow-release drug delivery system.

Collagen contains a number of available amino and hydroxy groups that may be used to bind the synthetic hydrophilic polymer. The polymer may be bound using a “linking group”, as the native hydroxy or amino groups that are present in collagen and in the polymer frequently require activation before they can be linked. For example, one may employ compounds such as dicarboxylic anhydrides (e.g., glutaric or succinic anhydride) to form a polymer derivative (e.g., succinate), which may then be activated by esterification with a convenient leaving group, for example, N-hydroxysuccinimide, N,N′-disuccinimidyl oxalate, N,N′-disuccinimidyl carbonate, and the like. See also Davis, U.S. Pat. No. 4,179,337, for additional linking groups. Presently preferred dicarboxylic anhydrides that are used to form polymer-glutarate compositions include glutaric anhydride, adipic anhydride, 1,8-naphthalene dicarboxylic anhydride, and 1,4,5,8-naphthalenetetracarboxylic dianhydride. The polymer thus activated is then allowed to react with the collagen, forming a collagen-polymer composition used to make the tubes.

For example, a pharmaceutically pure form of monomethylpolyethylene glycol (mPEG) (MW 5,000) may be reacted with glutaric anhydride (pure form) to create mPEG glutarate. The glutarate derivative is then reacted with N-hydroxysuccinimide to form a succinimidyl monomethylpolyethylene glycol glutarate. The succinimidyl ester (mPEG*, denoting the activated PEG intermediate) is then capable of reacting with free amino groups present on collagen (lysine residues) to form a collagen-PEG conjugate wherein one end of the PEG molecule is free or nonbound. Other polymers may be substituted for the monomethyl PEG, as described above. Similarly, the coupling reaction may be carried out using any known method for derivatizing proteins and synthetic polymers. The number of available lysines conjugated may vary from a single residue to 100% of the lysines, preferably 10-50%, and more preferably 20-30%. The number of reactive lysine residues may be determined by standard methods, for example by reaction with TNBS.

A number of sealants may be used in the present invention. In situ hydrogel forming compositions are known in the art and can be administered as liquids from a variety of different devices. One such composition provides a photoactivatable mixture of water-soluble co-polyester prepolymers and polyethylene glycol. Another such composition employs block copolymers of Pluronic and Poloxamer that are soluble in cold water, but form insoluble hydrogels that adhere to tissues at body temperature (Leach, et al., Am. J. Obstet. Gynecol. 162:1317-1319 (1990)). Polymerizable cyanoacrylates have also been described for use as tissue adhesives (Ellis, et al., J. Otolaryngol. 19:68-72 (1990)). WO 97/22371 describes two-part synthetic polymer compositions that, when mixed together, form covalent bonds with one another, as well as with exposed tissue surfaces. Similarly, U.S. Pat. No. 5,583,114 describes a two-part composition that is a mixture of protein and a bifunctional crosslinking agent has been described for use as a tissue adhesive. Particularly useful in the present invention are compositions that form a high strength medical sealant such as those described in U.S. Ser. Nos. 09/649,337 and 09/883,138. These compositions may include various collagenic materials (e.g., methylated collagen conjugated to PEG) as well as other tensile strength enhancers that impart the composition with a tensile strength comparable to that of cyanoacrylate adhesives. When one or more PEGs represents a component of the sealant, the PEG may be electrophilic or nucleophilic In addition, gelatinous, paste-like compositions may also be employed, since these forms tend to stay in place after administration more readily than liquid formulations. Preferred sealants for use in the present invention may exhibit resorption properties similar to that of the inventive stents and plugs. That is, the sealants may be resorbed by a patient as quickly as a needed for healing, e.g., typically about seven days, or as long as about 90 days. One of ordinary skill in the art will recognize that such sealants may also be provided as a powder or in another form on the surface of the inventive stents and plugs as discussed above.

A stent or plug according to the present invention may be produced in a number of ways. One simple method involves pouring a sterile stent solution into a sterile mold cavity to harden or cooling the stent solution until frozen. The mold cavity may be composed of stainless steel, elastomeric or thermoplastic tubing, glass, or other substances. Optionally, a releasing agent is interposed between the mold and the stent solution. A stent according to the present invention is preferably cast with hollow channels therethrough, but the plug is solid. Optionally, a stent according to the present invention is cast solid and bored to produce a hollow communication passage therethrough. A stent or plug according to the present invention is frozen through placement in a cryofreezer containing a stable temperature below about −40° C. or alternatively through immersion or thermal contact with a liquid nitrogen bath, or left to harden like wax. A stent or plug according to the present invention, upon removal from the mold, possesses a hard, glassy, or wax-like quality. Optionally, additives can be incorporated into a resorbable stent or plug prior to development or freezing. For example, an elasticizer such as glycerol may be added to physiologic saline solution before the solution is frozen to improve deformability of the frozen stent. Similarly, anticoagulant, such as heparin, may be incorporated into the inventive stent when the stent is employed in vascular anastomosis.

Extrusion may be employed as well to form the inventive stents and plugs. Most if not all of the above-described materials may be formulated for extrusion through a suitable orifice. Depending on the particular formulation, crosslinking may occur during or after extrusion. For example, a synthetic hydrophilic polymer is mixed with collagen. Within a relatively short period of time, the mixture is injected through a die, thereby forming a tube. In some instances, the mixture is allowed to gel or polymerize before injection to form covalent bonds between the polymer and the collagen and to increase the viscosity of the mixture for injection. Optionally, heat may be applied during extrusion to promote crosslinking such that the extruded tube does not collapse on itself.

In addition, a combination of selective crosslinking and pressurization may be employed to form the inventive stent. For example, tubular stents may be produced by mixing a collagen with a PEG. The collagen and polymer are mixed together thoroughly, the mixture is placed within a syringe and then injected from a wide-gauge needle of a syringe. The material is injected into a dilute solution containing a crosslinking agent, thereby forming a cylinder. The mixture is allowed to polymerize or crosslink within the solution for a period of time. Thereafter, the solid cylinder of material is removed from the solution, pressure is applied at one end, and the pressure is moved continuously towards the other end of the cylinder. This pressure causes unpolymerized material contained within the solid cylinder to be squeezed out of the solid cylinder, leaving a hollow opening, thus forming a tube. The tube can be dried by attaching both ends of the tube to supports and carrying out air-drying.

Generally, the microstructure of the stents should be controlled in order to produce a stent of controlled mechanical properties (e.g., tensile strength, elasticity) and resorption properties. For example, increasing the degree of crosslinking in the stent compositions tends to increase the stents' tensile strength, rigidity, and resistance to resorption. In addition, it is possible to use fibrillar and/or nonfibrillar collagen to form the stents of the invention. When microstructural uniformity is desired, nonfibrillar collagen such as gelatin may be employed. However, when microstructural anisotropy is desired, fibrillar collagen may be employed. In some instances, it may be desirable to align fibrils in the inventive stents and plugs to provide matrix directionality. For example, when a mixture of collagen and polymer is extruded from the orifice of an extrusion device, the fibers tend to orient along the direction of the injection. This orientation may impart additional tensile strength to the formed stents. In addition, this may influence the stents' rate of water uptake and/or resorbability. In addition, prior to casting or extrusion, it is important to control the void volume in the mixture. Typically, air bubbles are eliminated from the mixture before casting or extrusion, i.e., carry out deaeration. If air bubbles are trapped in the mixture, the bubbles may appear in the stents as breaks or weakened portions. On the other hand, a uniform dispersion of voids may enhance the resorption properties of the formed stents without introducing localized weak spots.

After a stent or plug is shaped, and polymerization has been completed, the stent may be dried. Drying can be accomplished in a variety of ways. For example, a tubular stent can be placed on a flat surface and exposed to the air and/or heat. Such a procedure tends to result in the flattening of the stent on the surface upon which the stent is placed. Further, there may be considerable overall shrinkage in stent length.

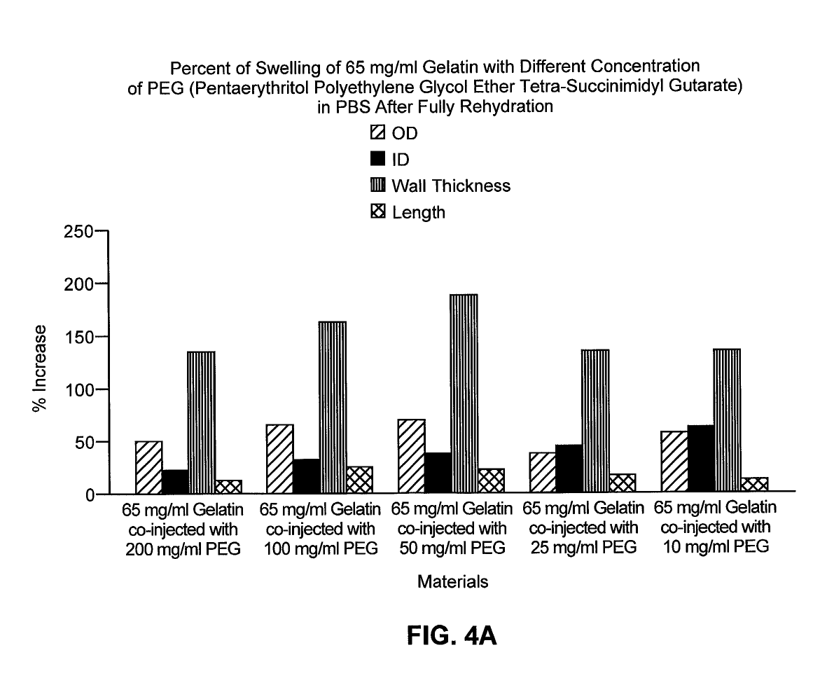

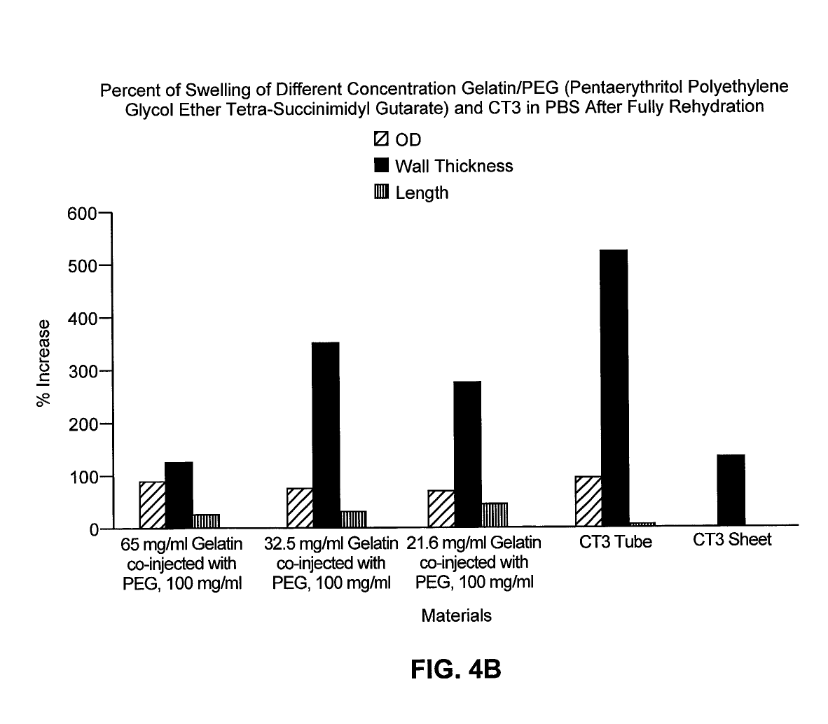

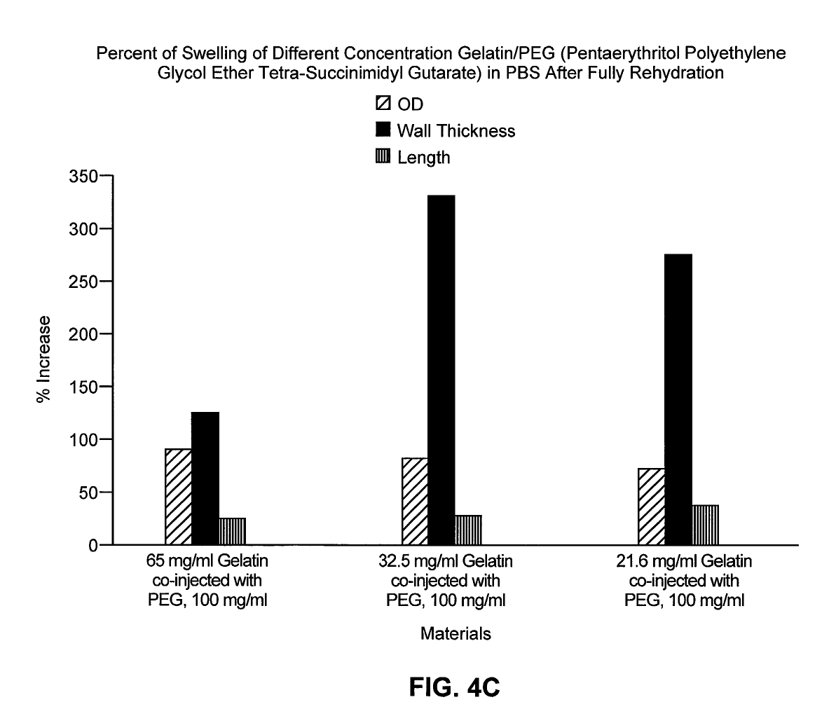

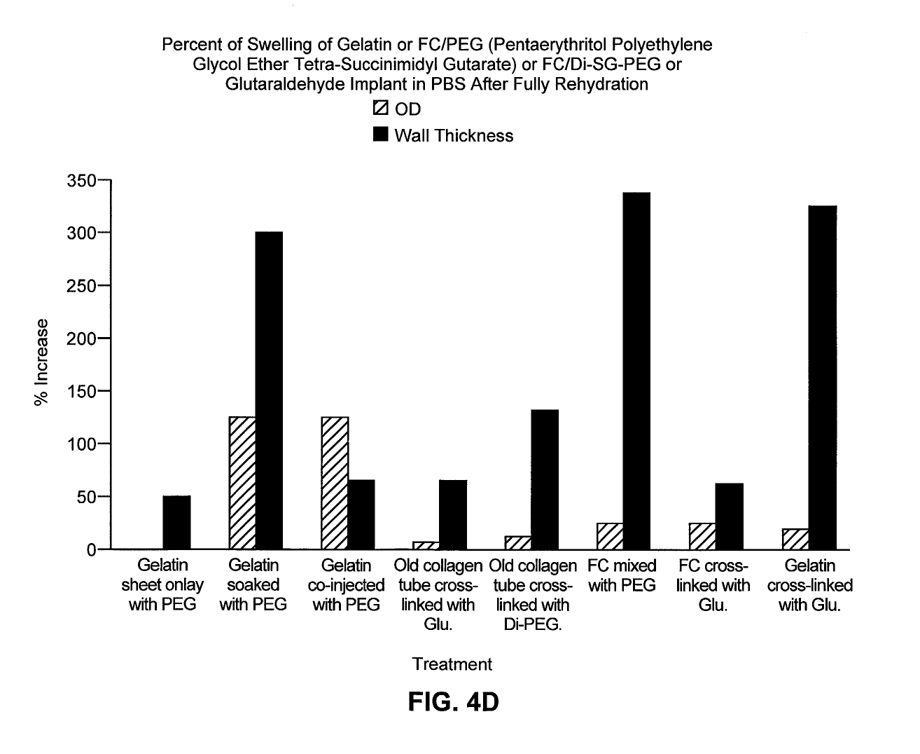

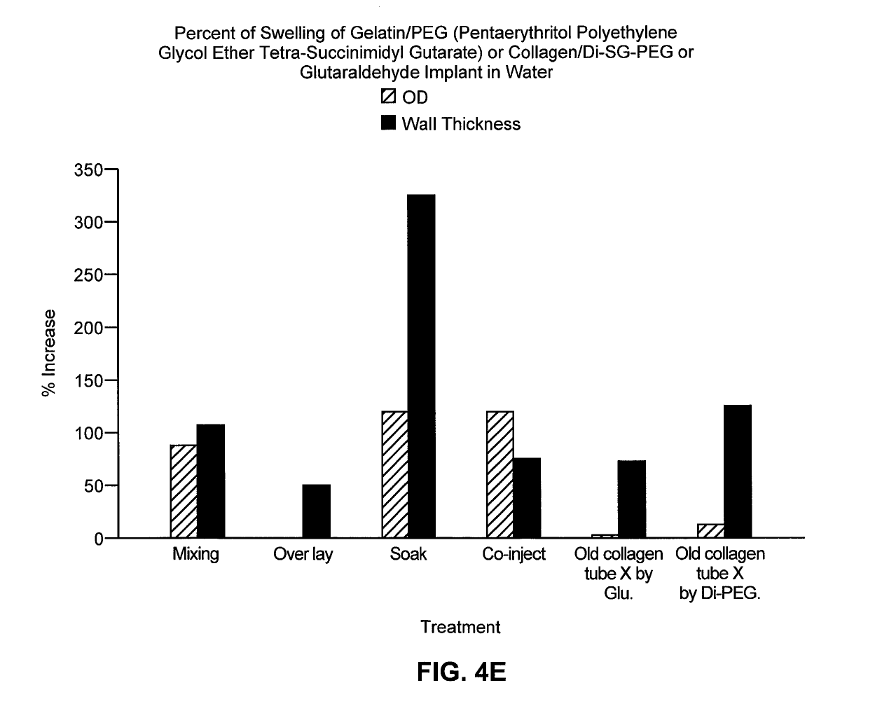

Since the inventive stents and plugs may expand in size upon hydration, it is generally preferable to store them in dehydrated form, and then hydrate them completely just prior to their insertion within a patient. By carrying out rehydration, the final size of the tube to be inserted can be precisely determined. It is also possible, however, to insert the stents and plugs in dehydrated form. For instance, a dehydrated stent may be inserted and slowly allowed to hydrate and expand 5-fold or more in situ, due to the presence of bodily fluids. Hydration rate can be increased, however, by injecting an aqueous solution into and around the stent. The aqueous solution may be a saline solution, or other salt-containing solution, in concentrations that match the surrounding environment—generally that of human tissue. Various resorbable prototype stents have been made from, e.g., PEG/collagen, PEG/gelatin, and gelatin cross-linked with glutaraldehyde; and their swelling behavior in a liquid such as phosphate buffered solution (PBS) has been characterized in FIG. 4. Pentaerythritol polyethylene glycol ether tetra-succinimidyl glutarate employed in these stents have an average molecular weight of 10,000 daltons. Swelling rate may correlate directly or inversely with resorption rate depending on the particular composition of the stent.

In addition, tensile testing of these stents has revealed that arteries joined with such stents combined with an adhesive may range in strength from about 1.3 to about 5.3 N/cm². However, by proper materials selection and application, tensile strength may be increased. Optimally, arteries or other blood vessels and tissues joined with such adhesives should either be comparable or exceed that resulting from a procedure employing Prolene® sutures comprising polypropylene or other threads made from synthetic or naturally occurring polymers.

Variations of the present invention will be apparent to one of ordinary skill in the art. For example, while particular attention has been given to PEG-collagen conjugates as a suitable material for forming the inventive stents and plugs, other conjugates, such as PEG-PEG and collagen-collagen, may be employed as well. Similarly, known surgical techniques that employ catheters and the like may be employed in conjunction with the inventive methods to carry out anastomosis. In addition, processing techniques may be combined to form the inventive articles. For example, after a stent is produced through extrusion, the stent may be cooled or frozen to render the stent more rigid for ease in manipulation.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

We claim:

1. An anastomosis stent for insertion into an opening in a lumen of a vessel or tissue of a patient, comprising:
a first terminus;
a second terminus;
an opening at each terminus; and
a primary lumen providing fluid communication between the openings at the first and second termini,
wherein at least one of the first and second termini is sized to be inserted into an opening in a vessel of a patient, and the stent is comprised of a non-polyglycolic acid material that is resorbable by the patient in about a few minutes up to about 90 days.

2. The stent of claim 1, wherein the primary lumen is substantially straight.

3. The stent of claim 1, wherein the primary lumen is curved, bent, or both.

4. The stent of claim 1, wherein at least one of the first and second termini is tapered or shaped.

5. The stent of claim 1, further comprising a flange at one of the first and second termini.

6. The stent of claim 1, wherein at least one of the first and second termini has a diameter of about 1 mm to about 10 mm.

7. The stent of claim 6, wherein the diameter is about 1 mm to about 8 mm.

8. The stent of claim 1, wherein the first and second termini have different diameters.

9. The stent of claim 1, wherein the termini are located about 1 cm to about 5 cm apart.

10. The stent of claim 9, wherein the termini are located at about 1.5 cm to about 4 cm apart.

11. The stent of claim 10, wherein the termini are located about 2 cm to about 3 cm apart.

12. The stent of claim 1, wherein at least one of the first and second termini is sized for anastomotic insertion into a blood vessel of the patient.

13. The stent of claim 12, wherein the blood vessel is an artery.

14. The stent of claim 13, wherein the artery is a coronary artery.

15. The stent of claim 13, wherein the artery is the patient's aorta.

16. The stent of claim 12, wherein the blood vessel is a vein of the patient.

17. The stent of claim 1, further comprising a third terminus and a third opening at the third terminus, wherein the third opening is in fluid communication with the primary lumen through an intersecting lumen.

18. The stent of claim 17, wherein the primary and intersecting lumens intersect at point closer to the first terminus than to the second terminus

19. The stent of claim 17 wherein the primary and intersecting lumens intersect perpendicularly.

20. The stent of claim 17, wherein the primary and intersecting lumens intersect non-perpendicularly.

21. The stent of claim 1, wherein the material is resorbable by the patient in about a few minutes to about ten days.

22. The stent of claim 21, wherein the material is resorbable by the patient in about seven days to about ten days.

23. The stent of claim 21, wherein the material is resorbable by the patient in about one day to about seven days.

24. The stent of claim 23, wherein the material is resorbable by the patient in about one day to about two days.

25. The stent of claim 1, wherein the material comprises frozen physiologic saline.

26. The stent of claim 1, wherein the material comprises a hydrophilic compound.

27. The stent of claim 1, wherein the hydrophilic compound comprises a polyethylene glycol-containing compound.

28. The stent of claim 27, wherein the polyethylene glycol-containing compound comprises a polyethylene glycol that is chemically conjugated with a naturally occurring compound.

29. The stent of claim 28, wherein the naturally occurring compound is a protein.

30. The stent of claim 29, wherein the protein is a collagenic material.

31. The stent of claim 30, wherein the collagenic material is a gelatin.

32. The stent of claim 30, wherein the collagenic material is selected from the group consisting of type I, type II, and type III collagens, and combinations thereof.

33. The stent of claim 27, wherein the naturally occurring compound is a polysaccharide.

34. The stent of claim 32, wherein the polysaccharide is selected from the group consisting of hyaluronic acid, cyclodextrin, hydroxymethylcellulose, cellulose ether, and starch.

35. The stent of claim 27, wherein the naturally occurring compound is a glycosaminoglycan or a proteoglycan.

36. The stent of claim 27, wherein the polyethylene glycol has a molecular weight of about 100 to about 20,000 daltons.

37. The stent of claim 26, wherein the hydrophilic material is comprises a collagenic material.

38. The stent of claim 37, wherein the collagenic material comprises a collagen that is chemically conjugated to a synthetic hydrophilic polymer.

39. The stent of claim 38, wherein the synthetic hydrophilic polymer is selected from the group consisting of polyethylene glycol and polyvinylpyrrolidone.

40. The stent of claim 1, further comprising a tissue sealant on a surface thereof.

41. A method of anastomosis comprising the step of:
(a) inserting the first terminus of the stent of claim 1 though an aperture into the cavity of a physiologically functioning vessel of a patient, and the second terminus of the stent into a conduit, such that an interface is formed between the vessel and the conduit about the aperture; and
(b) attaching the vessel to the conduit at the interface.

42. A method of anastomosis comprising the step of:
(a) inserting the first and second termini of the stent of claim 17 through in a physiologically functioning vessel of a patient, and the third terminus of the stent into a bypass conduit, such that an interface is formed between the vessel and the bypass conduit about the aperture; and
(b) attaching the vessel to the bypass conduit at the interface.

43. The method of claim 42, wherein step (b) is carried out without need for a suture.

44. The method of claim 42, wherein step (b) comprises (b′) introducing a tissue sealant around or over the interface between the vessel and the bypass conduit.

45. The method of claim 44, wherein the sealant comprises a collagenic material.

46. The method of claim 45, wherein the collagenic material comprises a methylated collagen.

47. The method of claim 45, wherein the collagenic material is selected from the group consisting of CIS, CSF, and combinations thereof.

48. The method of claim 44, wherein the sealant comprises a polyethylene glycol.

49. The method of claim 48, wherein the polyethylene glycol is selected from the group consisting of polyethylene glycol di-succinimidyl glutarate, pentaerythritol polyethylene glycol ether tetra-succinimidyl glutarate, pentaerythritol polyethylene glycol ether tetra-succinimidyl glutarate, polyethylene glycol mono-succinimidyl succinate, polyethylene glycol mono-succinimidyl propionic acid, polyethylene glycol mono-succinimidyl succinamide, polyethylene glycol di-succinimidyl succinamide, polyethylene glycol di-epoxide, polyethylene glycol di-isocyanate, polyethylene glycol di-carbonyldiimidazole, pentaerythritol polyethylene glycol ether tetra-maleimidopropionamide, pentaerythritol polyethylene glycol ether tetra-malimidopropionate, polyethylene glycol di-amine, diglycero polyethylene glycol ether tetra-amine, pentaerythritol polyethylene glycol ether tetra-amine, polyethylene glycol di-sulfhydryl, pentaerythritol polyethylene glycol ether tetra-sulfhydryl, pentaerythritol polyethylene glycol ether, diglycerol poly(ethylene glycol) ether, combinations thereof, and copolymers thereof

50. The method of claim 44, wherein step (b) further comprises, after step (b′), (b″) crosslinking the sealant.

51. The method of claim 44, wherein the tissue sealant is injected around or over the interface.

52. The method of claim 44, wherein the tissue sealant is applied as a spray.

53. The method of claim 42, wherein steps (a) and (b) are carried out simultaneously.

54. A tissue plug for use in sealing an opening in a patient's tissue, comprising a solid object having a platen surface, which is adapted to cover the opening, contact the perimeter about the opening, or both; wherein the solid object is comprised of a non-polyglycolic acid material that is resorbable by the patient in a maximum of about 90 days.

55. The plug of claim 54, further comprising a tissue sealant on a surface thereof.

56. The plug of claim 54, wherein the platen surface is supported by a pedestal structure having a pedestal lateral dimension.

57. The plug of claim 56, wherein the platen surface has a lateral dimension equal to the pedestal structure lateral dimension.

58. The plug of claim 56, wherein the platen surface has a lateral dimension greater than the pedestal structure lateral dimension.

59. The plug of claim 54, wherein the platen surface is nonplanar.

60. The plug of claim 54, wherein the platen surface is shaped to conform to the lumen surface of a blood vessel of the patient.

61. The plug of claim 60, wherein the blood vessel is an artery.

62. The plug of claim 61, wherein the artery is a coronary artery.

63. The plug of claim 60, wherein the blood vessel is the patient's aorta.

64. The plug of claim 54, wherein said resorbable material is selected from the group consisting of saline, polyethylene glycol, and blood plasma.

65. The plug of claim 54, wherein the material is resorbable by the patient in about one day to about ten days.

66. The plug of claim 65, wherein the material is resorbable by the patient in about seven days to about ten days.

67. The plug of claim 65, wherein the material is resorbable by the patient in about one day to about seven days.

68. The plug of claim 67, wherein the material is resorbable by the patient in about one to about two days.

69. The plug of claim 54, wherein the material comprises a hydrophilic compound.

70. The plug of claim 54, wherein the hydrophilic compound comprises a polyethylene glycol-containing compound.

71. The plug of claim 70, wherein the polyethylene glycol-containing compound comprises a polyethylene glycol that is chemically conjugated with a naturally occurring compound.

72. The plug of claim 71, wherein the naturally occurring compound is a protein.

73. The plug of claim 72, wherein the protein is a collagenic material.

74. The plug of claim 73, wherein the collagenic material is a gelatin.

75. The plug of claim 73, wherein the collagenic material is selected from the group consisting of type I, type II, and type III collagens, and combinations thereof.

76. The plug of claim 71, wherein the naturally occurring compound is a polysaccharide.

77. The plug of claim 76, wherein the polysaccharide is selected from the group consisting of hyaluronic acid, cyclodextrin, hydroxymethylcellulose, cellulose ether, and starch.

78. The plug of claim 71, wherein the naturally occurring compound is a glycosaminoglycan or a proteoglycan.

79. The plug of claim 70, wherein the polyethylene glycol has a molecular weight of about 100 to about 20,000 daltons.

80. The plug of claim 69, wherein the hydrophilic material is a collagenic material.

81. The plug of claim 80, wherein the collagenic material comprises a collagen that is chemically conjugated to a synthetic hydrophilic polymer.

82. The plug of claim 81, wherein the synthetic hydrophilic polymer is selected from the group consisting of polyethylene glycol and polyvinylpyrrolidone.

83. A method of sealing an opening in a patient's tissue comprising the steps of:
(a) positioning the plug of claim 54 in relationship to an opening in a patient's tissue, such that the plug covers the opening, contacts the perimeter about the opening, or both, thereby forming an interface between the plug and the tissue; and
(b) adhering the patient's tissue to the plug to form a closure.

84. The method of claim 83, wherein step (b) comprises (b′) introducing a tissue sealant around or over the interface.

85. The method of claim 84, wherein the sealant comprises a collagenic material.

86. The method of claim 85, wherein the collagenic material is a PEG-collagen.

87. The method of claim 84, wherein the sealant comprises polyethylene glycol.

88. The method of claim 84, wherein step (b) further comprises, after step (b′), (b″) crosslinking the sealant.

89. The method of claim 84, wherein the tissue sealant is applied through injection.

90. The method of claim 84, wherein the tissue sealant is applied as a spray.

91. The method of claim 83, wherein steps (a) and (b) are carried out simultaneously.

92. The method of claim 83, further comprising, after step (a), (b′) placing additional tissue in contact with the plug, such that the plug is interposed between the additional tissue and the tissue associated with the opening.

93. The method of claim 92, further comprising, after (b′), adhering the additional tissue to the tissue associated with the opening.

94. A sutureless method of anastomosis comprising the steps of:
(a) providing a stent comprising a first terminus, a second terminus, a third terminus, an opening at each terminus that fluidly communicate with each other through the interior of the stent, wherein the stent is comprised of a non-polyglycolic acid material that is resorbable by a patient in up to about 90 days;
(b) inserting the first and second termini of the stent though an aperture into a cavity of a physiologically functioning vessel of a patient, and the third terminus of the stent into a conduit, such that an interface is formed between the vessel and the by pass conduit about the aperture; and
(c) applying a tissue sealant at the interface to attach the conduit to the vessel.

95. A sutureless method of sealing an opening in a patient's tissue comprising the steps of:
(a) providing a plug comprised of a solid non-polyglycolic acid material that is resorbable by the patient in a maximum of about 90 days;
(b) positioning the plug in relationship to an opening in a patient's tissue, such that the plug covers the opening, contacts the perimeter about the opening, or both, thereby forming an interface between the plug and the tissue; and
(c) applying a resorbable sealant at the interface to form a closure.

96. A sutureless method of anastomosis comprising the steps of:
(a) providing a stent comprising a first terminus, a second terminus, a third terminus, an opening at each terminus that fluidly communicate with each other through the interior of the stent, wherein the stent is comprised of material that is resorbable by a patient in up to about 90 days;
(b) inserting the first and second termini of the stent though an aperture into a cavity of a physiologically functioning vessel of a patient, and the third terminus of the stent into a conduit, such that an interface is formed between the vessel and the by pass conduit about the aperture; and
(c) applying a tissue sealant at the interface to attach the conduit to the vessel such that the interface exhibits a tensile strength of at least about 1.3N/cm².
